(19) United States
(12) Patent Application Publication
(10) Pub. No.: US 2025/0041318 A1
(43) Pub. Date: Feb. 6, 2025

(54) STERILANTS COMPOSITION, KITS AND METHODS OF USE THEREOF

(71) Applicant: COMMUNITREAT LTD., Tel Aviv - Jaffa (IL)

(72) Inventors: Tami BARR, Meitar (IL); Hilik MAROM, Har Adar (IL)

(73) Assignee: COMMUNITREAT LTD., Tel Aviv - Jaffa (IL)

(21) Appl. No.: 18/715,728

(22) PCT Filed: Dec. 1, 2022
(86) PCT No.: PCT/IL2022/051283

§ 371 Date: Jun. 2, 2024

Related U.S. Application Data
(60) Provisional application No. 63/285,101, filed on Dec. 2, 2021.

(51) Int. Cl. A61K 31/585; A61K 31/11; A61K 45/06; A61P 15/16; A61P 15/18

(52) U.S. Cl. CPC A61K 31/585; A61K 31/11; A61K 45/06; A61P 15/16; A61P 15/18

(57) ABSTRACT

Provided a composition or a kit based on triptolide, gossypol and acceptable salts thereof. The current composition or kit based on triptolide, gossypol and acceptable salts thereof can be used for limiting or inhibiting birth rate including the induction of infertility.

### CROSS REFERENCE TO RELATED APPLICATIONS

This application claims the benefit of priority under 35 U.S.C. § 119 (e) of U.S. Provisional Patent Application No. 63/285,101, filed Dec. 2, 2021. The contents of the above applications are all incorporated by reference as if fully set forth herein in their entirety.

### FIELD OF THE INVENTION

The current disclosure relates generally to a composition, or a kit based on triptolide, gossypol and acceptable salts thereof. The current composition or kit can be used for limiting or inhibiting birth rate including the induction of infertility by combined inhibition of mitochondrial lactate dehydrogenase (LDH)—C₄ and nuclear factor κB(NF-κB) activity.

### BACKGROUND OF THE INVENTION

Population control of animals is a significant issue worldwide, and fertility regulation is necessary for the conservation of life supporting resources and animal welfare.

Sterilization of domestic animals has been applied for centuries to control the number of animals, genetic selection, tranquility of aggressive animals and to ensure the production of high-quality meat for human needs. The main methods of castration are operative castration, hormone inhibition, and chemical castration.

An ideal castration should be in such a way that it will provide permanent or continuous, low-cost treatment, block spermatogenesis and androgenetic and not affect animal welfare levels [Hassan, A., Fromsa, A. (2017). Review on Chemical Sterilization of Male Dogs. International Journal of Advanced Research, 5 (11), 758-770].

Surgical contraception by gonadectomy—surgical removal of either the testes in males or the ovaries in females, which results in a loss of gonadal production of sex steroids—is frequently instituted in animals and provides a permanent and irreversible effect. The procedure has several possible side effects such as underdevelopment of genital organs, disturbances in the musculoskeletal system, hormonal dysfunctions, urinary incontinence, risk of neoplasms, obesity, coat changes, etc. [Root Kustritz M. V., 2012. Effects of surgical sterilization on canine and feline health and on society. Reprod. Domest. Anim. 47, 214-222]. Yet, castration by open surgery requires post-operative care to minimize the risk of hemorrhage and infection. Besides, this method has some disadvantages: it is not cost-effective and time-consuming with risk of severe post-surgical complications. Non-surgical chemical sterilization has better advantages over other methods and among these, reduction of pain and stress, elimination of hemorrhage, hernia, infection, myiasis and other surgical sequelae.

In contrast to the surgical method, the challenge has been taken up by different reproductive biologists to develop a method of chemical sterilization, which may be a better alternative to surgical castration, as well as suited for mass-scale sterilization of male domestic animals without post-operative hazards. Different chemical agents were tried to bring about castration using inorganic chemicals, immune-contraceptives and hormones including androgen, progestogens, androgens plus progestogens and agonists for gonadotrophin releasing hormone (GnRH). Different researchers have evaluated non-surgical sterilization with injection of various hormones in many species of male animals, but these treatments failed to induce permanent sterility. Immunization techniques have also been used to induce antibodies against gonadotrophins and GnRH and had indicated that such immunization techniques vary in effectiveness and in duration of azoospermia. Adverse vaccination reactions were also observed as another disadvantage. Chemical sterilization has found application in some species of male animals such as monkeys, goats, bulls, hamsters, rabbits and dogs.

Hence, researchers over the past years have also tried various chemical agents such as danazol, glycerol, lactic acid, ferric chloride and ferrous sulphate, calcium chloride (CaCl2), bacillus calmette-guerin (BCG), zinc gluconate (Neutersol) and 20% hypertonic saline solution for induction of chemo-sterilization. However, all these chemical agents, following intratesticular injection had exhibited pain, pyrexia and even severe testicular inflammation (orchitis). Some agents such as, cadmium chloride, glycerol, lactic acid had caused selective destruction of testicular tissue with reversible testicular tissue damage. Even though the chemicals used had an effect on the destruction of testicular tissue, it had also complications and some drawbacks. For instance, in some cases, the interstitial portion of seminiferous tubules had regenerated after an initial phase of testicular atrophy and this had led to secondary male behavior causing management problems of the animals. Due to such type of complications caused by the use of the aforementioned chemicals, an effective chemo-sterilizing agent is yet to be established.

Several putative target proteins of triptolide have been reported, including polycystin-2, ADAM10, DCTPP1, TAB1, and XPB. Multiple triptolide-resistant mutations exist in XPB (ERCC3) and its partner protein GTF2H4. However, no triptolide-resistant mutations were found in polycystin-2, ADAM10, DCTPP1 and TAB1. Cys342 of XPB was identified as the residue that undergoes covalent modification by the 12,13-epoxide group of triptolide, and the XPB-C342T mutant rendered the T7115 cell line nearly completely resistant to triptolide. The level of resistance conferred by the C342T mutation is about 100-fold higher than the most triptolide-resistant mutants previously identified. Together, these results validate XPB as a target responsible for the antiproliferative activity of triptolide.

Minnelide is a more water-soluble synthetic analog of triptolide which is converted to triptolide in vivo. In a preclinical mouse model of pancreatic cancer, it was “even more effective than gemcitabine”.

Glutriptolide, a glucose conjugate of triptolide with better solubility and lower toxicity, did not inhibit XPB activity in vitro, but exhibited tumor control in vivo, which is likely due to sustained stepwise release of active triptolide within cancer cells.

The diterpene triptolide from Tripterygium wilfordii inhibits the production and gene expression of a range of cytokines and chemokines, including IL-1β, IL-6, TNF-α, and IFN-γ, in vitro, while it also decreases PGE₂ production via COX-2 gene suppression. Other mechanistic effects of triptolide include inhibition of NF-κB signaling via suppression of IκBα phosphorylation and it may modulate the T-cell response as it could also inhibit IL production in vitro. Furthermore, triptolide suppressed production and mRNA levels of promatrix metalloproteinase (MMP)-1 and -3 on human synovial fibroblasts induced by IL-1α, and cytokine-induced MMP-3, MMP-13 and aggrecanase-1 gene expression in chondrocytes and synovial fibroblasts.

Human lactate dehydrogenase (LDH) has five isoenzymes. Under anaerobic glucose conditions, pyruvate is reduced to lactate by LDH in the presence of NADH. Numerous reports suggest that the antifertility properties of gossypol are associated specifically with the (−)-isomer. (−)-Gossypol is a non-selective competitive inhibitor of NADH binding with LDH. It was attributed that its antifertility action to inhibition of mitochondrial LDH-C₄ (LDH-X), which is present only in the testes and sperm and is essential for energy production. However, the mode of action is complex and involves the inhibition of a number of essential enzyme systems, including ribonucleotide reductase, malate dehydrogenase (MDH), glyceraldehyde-3-phosphate dehydrogenase (GA₃PDH), and cytoplasmic phospholipase A₂ (cPLA₂). The latter enzyme plays an important role in the acrosomal reaction during sperm maturation. Genetic manipulation of cPLA₂α expression in rodents and cell culture models has led to a better understanding of how the enzyme influences cellular homeostasis and disease. cPLA₂α has an important role in embryo implantation and fertility. cPLA₂α has also been implicated in the pathophysiology of allergic inflammation, asthma, lung cancer metastasis, spinal cord injury, Alzheimer's disease and Niemann Pick type C cholesterol storage disorder. Inhibition of a recently discovered CIP transport protein caused increased cPLA₂α activity and eicosanoid production. Thus, the correlation between CIP levels and cPLA₂α activity provides evidence of a CIP/eicosanoid axis that contributes to the aforementioned diseases. cPLA₂₈ has been implicated in psoriasis but the role of other cPLA₂ isoforms in disease will require more investigative effort.

In the CNS, sPLA2 mRNA is expressed in response to the proinflammatory cytokines TNF-α, IL-1B, and IFN-γ. At the injury site, PLA₂ product, arachidonic acid is metabolized into proinflammatory metabolite such as prostaglandin E₂ (PGE₂), which not only facilitates macrophage and microglial recruitment but also increases local blood flow and enhances vascular permeability and proinflammatory cytokine expression.

Triptolide has a complex mechanism of action, which involves inhibition of transcription factor nuclear factor κB(NF-κB) activation, suppression of the production of prostaglandin E2, and reduction of cytokine levels. Triptolide was previously reported to inhibit the transcription factor, NF-κB. However, it is unclear whether the inhibition of NF-κB activation is linked to the inhibition of STAT 3 activation by triptolide. NF-κB and STAT 3 are activated by different cytokines; for example, tumor necrosis factor is a major activator of NF-κB, whereas IL-6 activates STAT 3. However, notably, JA K activation has been reported to regulate both STAT 3 and NF-κB activation (53). Therefore, triptolide is a potent chemotherapeutic agent targeting JA K regulation, which regulates NF-κB and STAT 3 activation. Notably, triptolide inhibits the Janus kinase (JA K)2 pathway in myeloproliferative disorder cells, which is a similar disease to MM (22).

Members of the JAK/STAT proteins, TYK 2, STAT 1, and STAT 4 are present and active in human sperm. The localization of STAT 1 and STAT 4 proteins to the apical region of the sperm head and their activation by IFN-α, IFN-g, or IL-12 implicate a role for sperm STAT proteins in fertilization. We hypothesize that sperm-derived phosphorylated STAT 1 and STAT 4 could contribute to the pool of transcription factors during sperm-oocyte fusion as well as transmit signal to the oocyte nucleus. Therefore, defects in sperm TYK 2 and STAT 1- or STAT 4-mediated signaling pathway may have relevance to male factor infertility.

Gossypol is a natural phenol derived from the cotton plant (genus Gossypium). Gossypol is a phenolic aldehyde that permeates cells and acts as an inhibitor for several dehydrogenase enzymes. It is a yellow pigment. The structure exhibits axial chirality, with the two enantiomers having different biochemical properties. Among other applications, it has been tested as a male oral contraceptive in China. In addition to its putative contraceptive properties, gossypol has also long been known to possess antimalarial properties.

The effect of gossypol acetic acid, a potent male sterilant was studied on LDH from goat liver (LDH-A₄), heart (LDH-B₄) and testis (LDH-C₄) in vitro. All the preparations of LDH were inhibited by gossypol when the reaction was carried out in pyruvate-lactate (direct) or lactate to pyruvate (reverse) directions. The IC₅₀ of gossypol for the pyruvate oxidation by LDH isozymes varied between 16 and 42 microM in presence of 0.27 mM pyruvate and 0.15 mM NADH at 25 degrees C. and pH 7.4 whereas for the lactate oxidation, IC₅₀ was 125 microM in a system containing 3.3 mM lactic acid and 1.8 mM NAD at 25 degrees C. and pH 9.0. Reciprocal plots due to Lineweaver-Burk showed that these isozymes are inhibited in a non-competitive manner with respect to pyruvate and lactate, and in a competitive fashion when NAD and NADH were varied as substrates. Ki values of LDH-A₄, —B₄ and —C₄ isozymes in presence of gossypol were 20, 34 and 29 microM against pyruvate; 33, 43 and 45 microM against NADH; 85, 85 and 125 microM against lactate and 94, 108 and 83 microM against NAD respectively.

However, traditional chemical sterilization requires trapping of the animal and treatment provided by a veterinarian or a specialized professional and releasing the animal thereafter. Moreover, in some cases more than one treatment is required. The whole procedure is time consuming and expensive and demands individual treatment and care.

### SUMMARY OF THE INVENTION

The following description is of the best currently contemplated modes of carrying out exemplary embodiments of the invention. The following description is not to be taken in a limiting sense but is made merely for the purpose of illustrating the general principles of the invention, since the scope of the invention is best defined by the appended claims.

In one embodiment, provided herein is a composition comprising: (a) triptolide, a triptolide prodrug, a pharmaceutically acceptable salt of triptolide, a pharmaceutically acceptable salt of a triptolide prodrug or any combination thereof; and (b) gossypol, a pharmaceutically acceptable salt of gossypol, or a combination thereof.

In a further embodiment, triptolide, triptolide prodrug, pharmaceutically acceptable salt of triptolide, pharmaceutically acceptable salt of triptolide prodrug, gossypol, pharmaceutically acceptable salt of gossypol, or any combination thereof is/are: extracted from a plant, synthetic or semi-synthetic.

In a further embodiment, a composition as described herein further comprises at least one additional active ingredient. In one embodiment, the additional active ingredient comprises an antibacterial agent, or an antiviral agent.

In a further embodiment, a composition as described herein is in an oral dosage form or an injectable dosage form.

In one embodiment, provided herein is a kit comprising: a first composition comprising: triptolide, a triptolide prodrug, a pharmaceutically acceptable salt of triptolide, a pharmaceutically acceptable salt of a triptolide prodrug or any combination thereof; and a second composition comprising gossypol, a pharmaceutically acceptable salt of gossypol, or a combination thereof.

In one embodiment, provided herein is a method for limiting birth, inducing contraception, inducing abortion, inducing sexual sterility, infertility, inhibiting in a subject, comprising administering to the subject, a therapeutically effective amount of the composition as described herein or the kit as described herein, thereby limiting birth, inducing contraception, inducing abortion, inducing sexual sterility or infertility, in a subject.

In a further embodiment, the subject is a mammal, a reptile, or a bird.

In a further embodiment, administering comprises contacting a reproductive organ with said composition.

In a further embodiment, the first composition and said second composition are administered separately or sequentially.

In a further embodiment, the first composition and said second composition are administered concomitantly.

In a further embodiment, administering comprises orally administering.

In a further embodiment, administering comprises administering to a reproductive organ.

In a further embodiment, the subject is a male. In a further embodiment, the subject is a female. In a further embodiment, the subject is a mammal or a bird.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts the suggested mechanisms of action in which triptolide and gossypol are active and their combined/synergetic activity. In FIG. 1: [1] Williams T J. Prostaglandin E2, prostaglandin I2 and the vascular changes of inflammation. Br J Pharmacol. 1979; 65 (3): 517-524. doi: 10.1111/j.1476-5381.1979.tb07860.x; [2] Akhlaq A. Farooqui. Molecular Aspects of Spinal Cord Injury. Ischemic and Traumatic Brain and Spinal Cord Injuries, 2018; [3] Fanny Odet, Chongwen Duan, William Willis, Eugenia Goulding, Aisha Kung, Mitch Eddy, Erwin Goldberg, Lactate Dehydrogenase-C₄ (LDH-C₄) Is Essential for Sperm Function., Biology of Reproduction, Volume 78, Issue Suppl_1, 1-5-2008, Page 187; [4] Kim J H, Park B. Triptolide blocks the STAT3 signaling pathway through induction of protein tyrosine phosphatase SHP-1 in multiple myeloma cells. Int J Mol Med. 2017 November; 40 (5): 1566-1572. doi: 10.3892/ijmm.2017.3122. Epub 2017-9-6. PMID: 28901387; [5] Keshmiri-Neghab H, Goliaei B. Therapeutic potential of gossypol: an overview. Pharm Biol. 2014 January; 52 (1): 124-8. doi: 10.3109/13880209.2013.832776. Epub 2013-9-30. PMID: 24073600; [6] Dodou K. (2005). Investigations on gossypol: Past and present developments. Expert Opin Investig Drugs 14:1419-34; [7] Dodou K, Anderson R J, Lough W J, et al. (2005). Synthesis of gossypol atropisomers and derivatives and evaluation of their antiproliferative and antioxidant activity. Expert Opin Investig Drugs 13:4228-37; [8] Chen S R, Dai Y, Zhao J, Lin L, Wang Y, Wang Y. A Mechanistic Overview of Triptolide and Celastrol, Natural Products from Tripterygium wilfordii Hook F. Front Pharmacol. 2018-2-14; 9:104. doi: 10.3389/fphar.2018.00104. PMID: 29491837; PMCID: PMC5817256; [9] Wu S, Divall S, Hoffman G E, Le W W, Wagner K U, Wolfe A. Jak2 is necessary for neuroendocrine control of female reproduction. J Neurosci. 2011-1-5; 31 (1): 184-92. doi: 10.1523/JNEUROSCI.2974-10.2011. PMID: 21209203; PMCID: PMC3079260; Sugita R, Kuwabara H, Sugimoto K, Kubota K, Imamura Y, Kiho T, Tengeiji A, Kawakami K, Shimada K. A Novel Selective Prostaglandin E2 Synthesis Inhibitor Relieves Pyrexia and Chronic Inflammation in Rats. Inflammation. 2016 April; 39 (2): 907-15. doi: 10.1007/s10753-016-0323-5. PMID: 26923147; Wei X, Gong J, Zhu J, Wang P, Li N, Zhu W and Li J: The suppressive effect of triptolide on chronic colitis and TNF alpha/TNFR2 signal pathway in interleukin 10 deficient mice. Clin Immunol 129:211 218, 2008; Qiu D and Kao P N: Immunosuppressive and anti inflammatory mechanisms of triptolide, the principal active diterpenoid from the Chinese medicinal herb Tripterygium wilfordii Hook. f. Drugs R D 4:1 18, 2003; Pavithra Ranganathan, et. al. Correlation of nuclear factor kappa B(NFKB) with sperm quality and clinical diagnoses in infertile men. Fertility and sterility. Vol. 78, S. 1, S95, Sep. 1, 2002.

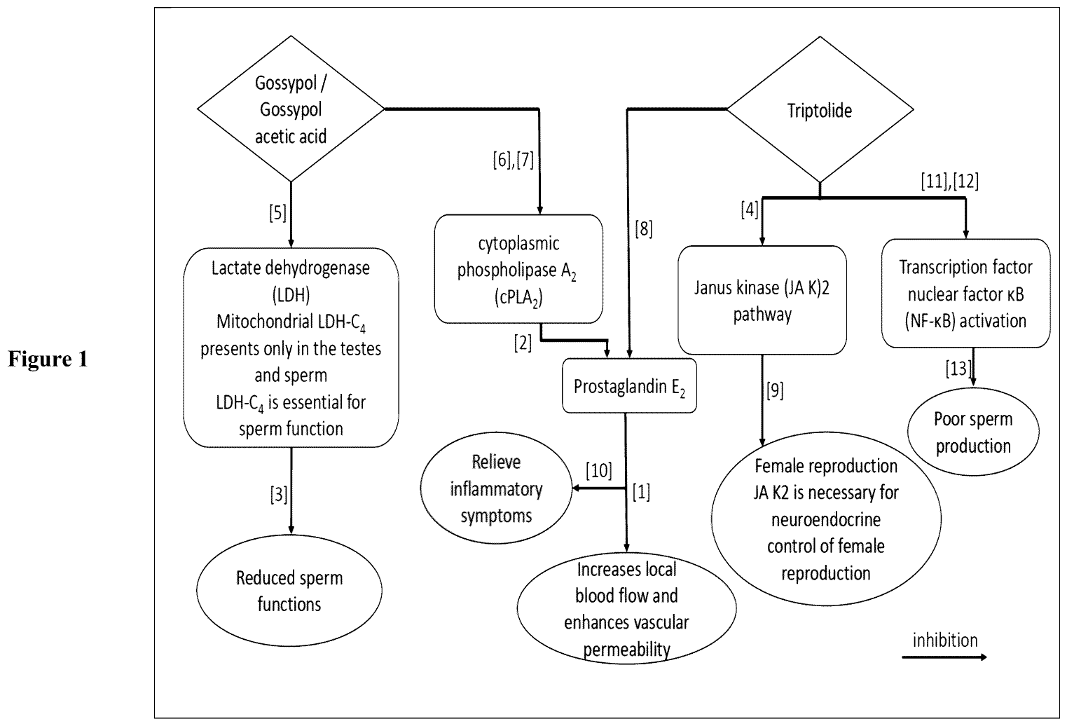

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one embodiment, provided herein a composition or a pharmaceutical composition comprising triptolide and/or its derivatives, salts, enantiomers, analogs or conjugates and gossypol and/or its derivatives, salts, enantiomers, analogs or conjugates. In one embodiment, a salt comprises a pharmaceutically acceptable salt.

The term “pharmaceutically acceptable salt” encompasses carboxylate salts having organic and inorganic cations, such as alkali and alkaline earth metal cations (for example, lithium, sodium, potassium, magnesium, barium and calcium); ammonium; or organic cations, for example, dibenzylammonium, benzylammonium, 2-hydroxyethyl ammonium, bis(2-hydroxyethyl) ammonium, phenylethylbenzylammonium, dibenzylethylenediammonium, and the like. Other cations encompassed by the above term include the protonated form of procaine, quinine and N-methylglucosamine, and the protonated forms of basic amino acids such as glycine, ornithine, histidine, phenylglycine, lysine, and arginine. The term “pharmaceutically acceptable salt” also includes salts formed by standard acid-base reactions with basic groups, such as amino groups, having a counterion derived from an organic or inorganic acid. Such acids include hydrochloric, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicylic, methanesulfonic, benzenesulfonic, sorbic, picric, benzoic, cinnamic, and the like.

In one embodiment, a “composition” as described herein comprises a pharmaceutical composition.

In one embodiment, provided herein a composition comprises: (a) a pharmaceutical effective amount of triptolide, a pharmaceutical effective amount of a triptolide salt, or a pharmaceutical effective amount of a combination thereof; and (b) a pharmaceutical effective amount of gossypol, a pharmaceutical effective amount of gossypol salt, or a combination thereof. In one embodiment, provided herein a composition comprises: (a) triptolide, a triptolide salt, or a combination thereof; and (b) gossypol, a gossypol salt, or a combination thereof.

The current invention is dealing with continuous oral administration of at least two active ingredients from botanical origin as described herein, for population control of patients, including birth control, contraception, sterility and infertility.

The current disclosure is dealing with combined inhibition of mitochondrial lactate dehydrogenase (LDH)—C₄ and nuclear factor κB(NF-κB) activity to reduce sperm quality in males and reduce fertility in females.

In one embodiment, a triptolide salt comprises Triptolide O-Methyl Phosphate Disodium Salt and 14-Succinyl triptolide sodium salt.

In one embodiment, a gossypol salt comprises Gossypol acetic acid salt and Gossypol Sodium Salt.

In one embodiment, a triptolide comprises a triptolide prodrug, or a pharmaceutically acceptable salt thereof, having a structure selected from:

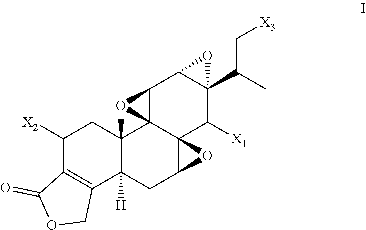

where X¹ is OH or OR¹, and X² and X³ are independently OH, OR¹ or H, with the proviso that at least one of X¹, X² and X³ is OR′, and at least one of X² and X³ is H; and R¹ is —C(O)—Y—Z, wherein Y is a branched or uribranched C₁-C₆ alkyl or alkenyl chain; and Z is COOR², NR³R³′, or ⁺NR⁴R⁴′R⁴″, where R² is a cation; R³ and R³′ are independently H or branched or unbranched C₁-C₆ alkyl, hydroxyalkyl, or alkoxy-alkyl, or R³ and R³′ taken together form a 5- to 7-member heterocyclic ring whose ring atoms are selected from the group consisting of carbon, nitrogen, oxygen and sulfur, wherein said ring atoms include 2 to 6 carbon atoms, one or more nitrogen atoms, and optionally one or more oxygen or sulfur atoms, and said ring is unsubstituted or is substituted with one or more groups selected from R⁵, OR⁵, NR⁵R⁶, SR⁵, NO₂, CN, C(O)R, C(O)NRR⁶, OC(O)R⁵, OC(O)NRR⁶, and halogen, where n and R⁶ are independently hydrogen, lower alkyl or lower alkenyl; and R⁴, R⁴′, and R⁴″ are independently branched or unbranched C₁-C₆ alkyl, hydroxyalkyl, or alkoxyalkyl;

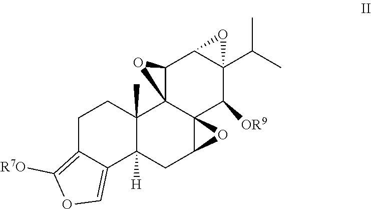

where OR⁷ is selected from (i) a carboxylic ester, carbonate, or inorganic ester, having a central atom selected from carbon, sulfur, phosphorus, nitrogen, and boron, and having linked to said central atom at least one group of the form —Y—Z′ or —O—Y—Z′, where Y is a branched or unbranched C₁-C₆ alkyl or alkenyl chain, and Z′ is hydrogen or a polar group selected from keto, aldehyde, carboxylate, carboxylic ester, hydroxy, alkoxy, polyether, thiol, alkylthio, amino, cyano, nitro, sulfate, nitrate, phosphate, or a 5- to 7-membered heterocycle having ring atoms selected from carbon, nitrogen, oxygen, and sulfur, and three to six carbon ring atoms, and (ii) a mono-, di- or trisaccharide linked to C14 at an anomeric center; and OR⁹ is OH or O—(C═O)R, where R is lower alkyl;

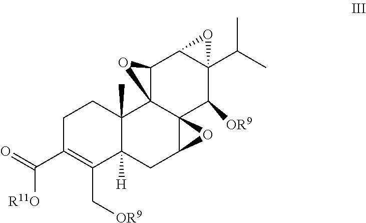

where OR¹¹ is selected from (i) —O—Y—Z or —O—(C═O)—Y—Z, where Y is a branched or unbranched C₁-C₆ alkyl or alkenyl chain, and Z is hydrogen or a polar group selected from keto, aldehyde, carboxylate, carboxylic ester, amino, alkylamino, hydroxy, alkoxy, polyether, thiol, alkylthio, cyano, nitro, inorganic ester, or a 5- to 7-member heterocyclic ring whose ring atoms are selected from the group consisting of carbon, nitrogen, oxygen and sulfur, where the ring atoms include 3 to 6 carbon atoms, and (ii) a mono-, di- or trisaccharide linked to C₁₄ at an anomeric center; and OR⁹ is —O—(C═O)R, where R is lower alkyl; and

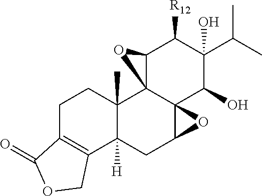

where R¹² is a leaving group selected from the group consisting of alkyl sulfonate, fluoroalkyl sulfonate, aryl sulfonate, fluorosulfonate, nitrate, alkyl phosphate, alkyl borate, trialkylammonium, and dialkylsulfoniumn; and OR⁹ is OH or O—(C═O)—R, where R is lower alkyl.

“Alkyl” refers to a fully saturated acyclic monovalent radical containing carbon and hydrogen, which may be branched or a straight chain. Examples are methyl, ethyl, n-butyl, t-butyl, n-heptyl, and isopropyl. “Cycloalkyl” refers to a fully saturated cyclic monovalent radical containing carbon and hydrogen, which may be further substituted with alkyl. Examples are cyclopropyl, methyl cyclopropyl, cyclobutyl, cyclopentyl, and cyclohexyl. “Lower alkyl” refers to an alkyl radical of one to six carbon atoms, preferably one to four carbon atoms, as exemplified by methyl, ethyl, n-butyl, i-butyl, t-butyl, isoamyl, n-pentyl, and isopentyl.

“Alkenyl” refers to a monovalent or divalent unsaturated, preferably mono-unsaturated, radical containing carbon and hydrogen, and which may be cyclic, branched or a straight chain. “Lower alkenyl” refers to such a radical having one to four carbon atoms.

“Acyl” refers to a radical having the form —C(O)R, where R is an alkyl, aryl, or an aralkyl group.

In one embodiment, a triptolide comprises the compound represented by:

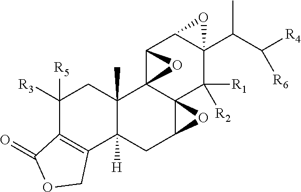

where: R¹ is H or R, where R is selected from lower alkyl, alkenyl, alkynyl, and allenyl, or R¹ together with R²═O (oxo); R²—OH or R1 and R² together-O (oxo); CR3R5 and CR4R6 are selected from CH2, CHOH and CROH; at least one of R1, R5 and R6 is R; and at least one of CR3R5 and CR4R6 is CH2.

“Aryl” refers to a substituted or unsubstituted monovalent aromatic radical having a single ring (e.g., benzene) or two condensed rings (e.g., naphthyl). This term includes heteroaryl groups, which are aromatic ring groups having one or more nitrogen, oxygen, or sulfur atoms in the ring, such as furyl, pyrrole, pyridyl, and indole. By “substituted” is meant that one or more ring hydrogens in the aryl group is replaced with a halide such as fluorine, chlorine, or bromine; with a lower alkyl group containing one or two carbon atoms; nitro, amino, methylamino, dimethylamino, methoxy, halomethoxy, halomethyl, or haloethyl.

“Aralkyl” refers to an alkyl, preferably lower alkyl, substituent which is further substituted with an aryl group; one example is a benzyl group.

A “heterocycle” refers to a non-aromatic ring, preferably a 5- to 7-membered ring, whose ring atoms are selected from the group consisting of carbon, nitrogen, oxygen and sulfur. Preferably, the ring atoms include 3 to 6 carbon atoms. Such heterocycles include, for example, pyrrolidine, piperidine, piperazine, and morpholine.

In one embodiment, triptolide and/or its derivatives, salts, enantiomers, analogs or conjugates is/are extracted from a plant source. In one embodiment, triptolide and/or its derivatives, salts, enantiomers, analogs or conjugates is/are synthetic.

In one embodiment, gossypol and/or its derivatives, salts, enantiomers, analogs or conjugates is/are extracted from a plant source. In one embodiment, gossypol and/or its derivatives, salts, enantiomers, analogs or conjugates is/are synthetic. In one embodiment, gossypol comprises a co-crystals of (−)-gossypol. In one embodiment, gossypol comprises a co-crystals of (−)-gossypol with acetic acid.

In one embodiment, gossypol comprises Gossypol acetate, Apo gossypol, levorotation gossypol, Apo gossypol ketone ApoG2, gossypol ketone, Methyl gossypol, gossypol schiff bases, monoaldehyde gossypol, acetyl gossypol, monoether gossypol or any combination thereof.

In an embodiment of the invention, the composition further comprises at least one additional active ingredient. In an embodiment of the invention, the at least one additional active ingredient is of plant source. In an embodiment of the invention, the additional active ingredient is a selected extract from Momordica charantia, Aspilia fricana, Rivea hypocrateriformis, Cissampelos pareira, Curcuma longa, Acacia leucophloea, Butea monosperma, Piper bitle, Cassia fistula, Ocimum gratissimum, Ficus religiosa, Calotropis procera, Terminalia belerica, Physalis alkekengi, Leonotis ocymifolia, Alianthus excelsa, Atrabotrys odoratissimus, Coriandrum sativum, Melia azedarach, Trianthema portulacastrum, Balantis roxburghii, Cannabis sativa, Carica papaya, Hibiscus rosasinensis, Andrographis paniculate, Tripterygium wilfordii, Solanum surattense, Embelia ribes, Stephania hernandifolia, Catharanthus roseus, Abrus precatorius, Azadirachta indica, Aegle marmelos or Apium graveolens. In an embodiment of the invention, the additional active ingredient is a selected from Kuguacins F—S; β-sitosterol; Daucosterol; Campesterol; Stigmasterol; β-sitosterol; ξ-isopropenylchole-5, (6)-ene-3-O-β-D-lucopyranoside; Diosgenin; Δ5-avenasterol; 25,26-dihydroelasterol; 3-[(5β,19-Epoxy-19,25-dimethoxycucurbita-6,23-dien-3-yl)-2-oxoacetic acid; 3-[(5B,19-Epoxy-19,25-dimethoxycucurbita-6,23-dien-3-yl)oxy]-3-oxopropanoic acid; 3-[(5-Formyl-7β-hydroxy-25-methoxycucurbita-5,23-dien-3-yl)-oxy]-3-oxopropanoic acid; 3-[(5-Formyl-7βmethoxy-7,23S-dimethoxycucurbita-5,23-dien3-yl)oxy]-3-oxopropanoic acid; 3-[(25-O-Methylkaravilagenin D-3-yl)oxy]-2-oxoacetic acid; 3-[(5-Formyl-7β,25-dihydroxymethoxycucurbita-5,23-dien-3-yl)-oxy]-3-oxopropanoic acid; palmitic; stearic; pentadecanoic; arachidic; linoleic; capric; palmitic; oleic; heneicosanoic; α-linolenic; nonadecanoic; tridecanoic; decanoic; lauric; palmitoleic; heptadecanoic; docosanoic; tetracosanoic; tridecanoic; myristic; behenic; lignoceric; α-eleostearic; gallic; caffeic and ellagic; chlorogenic; protocatechuic; tannic; p-hydroxylbenzoic; vanillic; chlorogenic; p-coumaric; ferulic; 3-coumaric; 4-coumaric; o-coumaric; mcoumaric; p; βresorcylic; vanillic; syringic; gentisic; salicylic; veratric; tcinnamic and homogentisic acids; epigallocatechin; gallocatechin gallate; quercetin; luteolin-7-O-glycoside; naringenin7-O-glycoside; apigenin-7-O-glycoside; myricetin; quercetin; kaempferol; luteolin; apigenin; hesperidin; naringenin; biochanin a; naringin; catechin; epicatechin; quercitrin; isoquercitrin; quercetin and kaempferol; α-pinene; phytol; cannabidiol, β-caryophyllene; carene; germacrene D; D-limonene; 1-methoxy-4-(2-propenyl)benzene; D-(+)-carvone; 6-methyl-2,4-di-t-butylphenol; humulene; 1-allyl-2,5-dimethoxy-3,4-methylenedioxybenzene (diplaniol); heptadecane; eicosane; n-heneicosane; docosane; tricosane; n-pentacosane; dioctylester of 1,2-phenyldicarboxylic acid; octacosane; n-nonacosane; (+)-s-methyl-1-cysteine sulfoxide(methiin); (+)-s-ethyl-1-cysteine sulfoxide (ethiin); (+)-s-propyl-1-cysteine sulfoxide (propiin); (+)-s-allyl-1-cysteine sulfoxide (alliin); (+)-s-(trans-propenyl)-1-cysteine sulfoxide (isoalliin); (+)-s-butyl-1-cysteine sulfoxide (butiin); (SSRC)—S-(3-pentenyl)-L-cysteine sulfoxide; S-methylcysteine; S-ethylcysteine; S-propylcysteine; S-allyl-cysteine; S-2-hydroxyethyl-cysteine; S-propylmercapto-cysteine; Reticuline; Isococlaurine; Laudanosine; Warifteine; Des-7′-O-methylroraimine; Epi-Des-7′-Omethylroraimine; Insulanoline; Insularine; Cycleanine; Cissacapine; Cissampareine; (−)-Curine; R,S-tubocurine; S,R-tubocurine; Hayatinine; R,S-12-O-methyl-curine; Tobocurane; Isochondodendrine; Isoliensinine; Roraimine; Pelosine; Dihydrowarifteine; Dimethyldihydrowarifteine; Methyldihydrowarifteine; Dimethylwarifteine; (+)-Bebeerine; Daijisong; Cissampentine; Simpodialine-b-N-oxide; Bulbocapnine; (+)-Dicentrine; Lauroscholtzine; Liriodenine; Cissaglaberrimine; Laurifoline; Magnoflorine; Oxobuxifoline; Nuciferine; (+)-Corytuberine; Dihydrodicentrine; (+)-Corydine; Milonine; Salutaridine; Steponine; (−)-Cissamine; (−)-b-cyclanoline; Isoimerubrine; Pareirubrine A; Pareirubrine B; Grandirubrine; Pareitropone; Norimeluteine; Norruffscine; Eletefine; Pronuciferine; Glaziovine; Crotsparine; Sepeerine; (−)-Oblongine; Magnocurarine; (+)-Obaberine; (+)-Obamegine; (+)-Homoaromoline; (−)-Nor-NO-chondrocurine; (+)-Tetrandrine; kaempferol 3-O-b-D-glucopyranoside; kaempferol 3-O-bD-glucuronopyranoside; quercetin 3-O-sophoroside [quercetin 3-OD-glycosyl-(1,2)-D-glucoside]; naringenin 7-ODglucoside; eriodictyl-7-O-β-D-glucoside; galangin7-glucoside; baicalein-7-O-glucoside; trans-N-feruloyltyramine; Cissampeline; Ar-curcumene; Zingiberene; Bisabolene; Sesquiphellandrene; Benzene; 1-Ethyl-4-isobutylbenzene; Ar-tumerone; Curlone; Benzaldehyde; Silane; 1,2,3,5-tetramethyl-Benzene; Phenol; 4-Methyl-carbanilonitrile; β-Myrcene; Tricyclo[2.2.1.0 (2,6) heptane; 3-trimethyl; 3-Carene; α-Terpinen; o-Cymene; Eucalyptol; p-Mentha-1,4 (8)-diene; β-Linalool; p-Cymen-8-ol; α-Caryophyllene; α-Curcumene; β-Sesquiphellandrene; Nerolidol; 1H-Indene,2,3,3a,4,7,7a-hexahydro2,2,4,4,7,7-hexamethyl; 5H-1,4-Dioxepin, 2,3-dihydro-2,5-dimethyl; Ethyl trans-3-methyl-2-oxiranecarboxylate 4H-Pyran-4-one, 2,3-dihydro-3,5-dihydroxy-6-methyl-3-Acetyl-2-octanone; Tetradecane; 2-methoxy-4-vinylphenol; 1,4-dimethylpiperazine; Octadecane; Phenol,2,4-bis(1,1-dimethylethyl)-2,3-dihydroxycyclohexanone; Dodecanoic acid; 4-methyl-2,5-dimethoxybenzaldehyde; Fumaric acid, ethyl 2-methylallyl ester; Megastigmatrienone; Docosane; 4-((1E)-3-Hydroxy-1-propenyl)-2-methoxyphenol; beta-k-Strophanthin; Tetradecanoic acid; 3-(3-Oxo-tetrahydro-pyran-2-yl)-propionic acid; (−)-Loliolide; Salicin; 3-heptadecanol; Isopropyl myristate; 2-methyl-2-(4h-1,2,4-triazol-4-ylamino) propanenitrile; Neophytadiene; 2-Pentadecanone; trimethyl; Mome inositol; (E)-phytol; Hexadecanoic acid; methyl ester; Cyclohexanone; 2,6-bis(2-methylpropylidene); 2 3-ethyl-3-undecanol; N,n-bis(2-hydroxyethyl) dodecanamide; α-d-glucopyranoside, methyl; Methyl octadeca-9,12-dienoate; 9,12,15-octadecatrienoic acid; methyl ester; Undecanoic acid 9,12,15-Octadecatrienoic acid; Octadecanoic acid; 9-Octadecenoic acid; phenylmethyl ester; Diethylheptadecane; α-amyrin acetate; Butin; palmitine hydroxide; Caption; α-Thujeneb; γ-Cadineneb; Linalool; Decanal (capric aldehyde); Hexadeconoic acidb; 1,8-cineolb; Eugenolb; Methyl eugenolb; Eugenol acetate; Campheneb; A-cadinene; α-Terpineol; Decanal (laural aldehyde); Terpinyl acetate; Caryophyllene oxide; Iso-eugenol; Methyl chavicol; Methyl benzoate; Sabineneb; α-Cadinene; Terpinol-1-ol; Stearaldehyde; Chlorogenic acid; Chavicol; Anethole; Methyl salicylate; Trans-sabinene hydrate; β-Salinene; α-Costol; n-Decanole; Chavibetol; Safrole; Chavibetol acetate; β-Myrcene; β-Slemene; Δ-Cardinol; Hydroxy-chavicol; Allylpyrocatecol diacetate; Trans-β-ocimene; γ-Elemene; 3,7,11,15-tetra-methyl-2-hexadecane-ol; Bornylene; Cis-caryophyllene; Geraniolb; β-Pinene; Trans-Caryophyllene; α-Cardinole; Trans-β-ocimene; Aromadendrene; τ-Muurolole; γ-Terpenine; α-Cubebene; α-Selinenolf; Terpinolene; β-Cubebene; Allo-ocimene; α-Humulene; α-Terpenene; γ-Muurolene; β-Phellandrene; Germacrene D; Limonene; Lepidozene; p-cymene; 2,6,6-Trimethyl-1-methyl-cyclo-hex-2-ene; α-Pinene; eugenol; 1,8-cineole; m-xylene; 1,2,4-Trimethylcyclohexane; o-xylol Phenolic Compound; p-xylol Phenolic Compound; Isopropyl benzene; m-Tolualdehyde; Quercetin; 6,10,14-Trimethyl pentadecanone-2-one; n-Eicosane; (E)-S-Eicosene; 3,6,6-Trimethyl-2-norpinanol; 3-Pentadecyl valerate; n-Hexdec-2-en-1-ol; 1,2,3,4,5-Pentametyl cyclopentane; Tetratriacontyl hepta flurobutyrate; Kaempferol; Gallotannin; Elligatannin; Gallic acid; Chebulic acid; Chebulagic acid; Chebulinic acid; Ellagic acid; Anthrquinone glycosides; Punicalagin ((2,3-(S)-hexahydroxydiphenoyl-4,6-(S, S)-gallagyl-D-glucose); terflavin A; Terchebulin; Terchebin (1,3,6-trigalloyl glucose,); Terflavins B; Terflavin C; Terflavin D; Punicalin; Neo-chebulic acid; 1,6-di-O-galloyl-D-glucose; Gallic acid (3,4,5-Trihydroxybenzoic acid); Casuarinin; Chebulanin; Corilagin; Ellagic acid (2,3,7,8-Tetrahydroxy-chromeno [5,4,3-cde]chromene-5,10-dione); Chebulagic acid; Chebulinic acid (1,3,6-Tri-O-galloyl-2,4-chebuloyl-β-D-glucopyranoside); 1,2,3,4,6-penta-O-galloyl-D-glucose; 2,3,4,6-tetra-O-galloyl-β-D-glucose; Ethyl gallate (Ethyl 3,4,5-trihydroxybenzoate); Methyl gallate (Methyl-3,4,5-trihydroxybenzoate); Chebulaginic acid; 4-O-methylgallic acid; Methyl(S)-flavogallonate; Methyl neochebulagate; Eugenol; Ascorbic acid; Triethyl chebulate; Tannic acid [2,3-dihydroxy-5-({[(2R,3R,4S,5R,6R)-3,4,5,6-tetrakis ({3,4-dihydroxy-5-[(3,4,5-trihydroxyphenyl)carbonyloxy]phenyl}carbonyloxy) oxan-2-yl]methoxy}carbonyl)phenyl 3,4,5-trihydroxybenzoate]; 2,4-Chebulyl-beta-D-glucopyranose; Shikimic acid; Ferulic acid; Vanillic acid; p-Coumaric acid; Caffeic acids; Melilotic acid; Phloroglucinol [benzene-1,3,5-triol]; Pyragallol [1,2,3-Trihydroxybenzene]; Phenol; Arjungenin; Arjunolic acid; Arjunic acid; Terminolic acid; Arjunglucoside I; Arjunglucoside II; Arjunetin; Chebuloside II; Bellericoside; Chebuloside I [2a, 3B, 23-Trihydroxy-olean-12-en-28-oic; Acid]; 2α-Hydroxyursolic acid; 2α-Hydroxymicromiric acid; Maslinic acid [(4aS,6aR, 6aS, 6bR, 8aR, 10R, 11R, 12aR, 14bS)-10,11-dihydroxy-2,2,6a, 6b, 9,9,12a-heptamethyl-1,3,4,5,6,6a,7,8,8a,10,11,12,13,14b-tetradecahydropicene-4a-carboxylic acid]; β-caryophyllene; α-Phellandrene [a: 2-Methyl-5-(1-methylethyl)-1,3-cyclohexadiene]; α-Terpinene; Terpinen-4-ol; Terpinolene; Chebupentol [Olean-12-ene-2,3,19,23,28-pentol, (2a, 3b, 4a, 19a)]; Rutin; Quercetin; Luteolin [2-(3,4-Dihydroxyphenyl)-5,7-dihydroxy-4-chromenone]; Isoquercetin [2-(3,4-Dihydroxyphenyl)-5,7-dihydroxy-3-[(2S,3R,4S,5S,6R)-3,4,5-Trihydroxy-6-(hydroxymethyl) oxan-2-yl]oxychromen-4-one]; 3′-Methoxy quercetin; 3,4-Dimethoxy quercetin [5,7-Dihydroxy-2-(3-hydroxy-4-methoxyphenyl)-3-Methoxy-4H-chromen-4-one]; Pelargonidin; β-Sitosterol; Daucosterol; Behenic acid [Docosanoic acid]; Stearic acid [Octadecanoic acid]; Palmitic acid [hexadecanoic acid]; Oleic acid [(9Z)-Octadec-9-enoic acid]; Arachidic acid [icosanoic acid]; Linoleic acid [9Z, 12Z)-9,12-Octadecadienoic acid]; 12-Hydroxyoctadec-cis-9-enoic acid (ricinoleic acid); 2-Undecanone; Cyclododecane; 9-Octadecene; Hexadecane; Cylohexane; 8-Pentadecanone; 9-Eicosene; Triacontane; Tetradecane; Oxirane; 1,16-Hexadecanediol; Heptylcyclohexane; 10-Nonadecanone; Phthalic acid; Tritetracontane; 9-Heptadecanone; Tetratetracontane; Linoleic acid ethyl ester; 9-Octadecenoic acid ethyl ester; 9,12,15-Octadecatrienoic acid; 1-Tricosene; 1,19-Eicosadiene; Heptafluorobutyric acid; 1-Octanol; 1-Decanol; Cyclooctacosane; 1H-Indene; Hexacosyl pentafluoropropionate; Octatriacontyl pentafluoroprppionate; Tetratriacontane; 1,2-benzenedicarboxylic acid; Ibogamin-9 (17H)-ol [(9a)-12-Methoxy-16,17-didehydro-9,17-dihydroibogamin-9-ol]; 9-Tricosene; Tetratriacontyl heptafluorobutyrate; Dotricontyl heptafluorobutyrate; Tetracosanoic acid [Lignoceric acid]; Pentatriacontane; Eicosyl trifluoroacetate; Squalene; Tetracosyl heptafluorobutyrate; Tetratriacontyl heptafluorobutyrate; Heptafluorobutyric acid; Sulfurous acid; Octacosanoic acid; Vitamin E; Tetracosyl heptafluorobutyrate; Hexacosanoic acid; Octatriacontyl pentafluoropropionate; Triacontanoic acid [Melissic acid]; Tricosyl pentafluoropropionate; Acetic acid; megestrol acetate; Heptacosanoic acid; Tetratriacontyl pentafluoropropionate; Tetracosanoate; Kaempferol-3-rutinoside; Ethanedioic acid; physalin P; 4,7-didehydroneophysalin B; physalin D; 5α-hydroxy-25,27-dihydro-7-dehydro-7-deoxyneophysalin A; 4,7-didehydrophysalin B; ursolic acid; wogonin; blumenol A; nobiletin; liquiritigenin; schizandrin; 5-hydroxymethylfurfural; 5-(hydroxymethyl)-2-(dimethoxymethyl) furan; 1-O-[3-O-2-methyl-5-(2,3,4-trimethyl)phenyl-2,3-pentanediol]-β-D-xylopyranosyl-(1→6)-β-D-galactopyranoside; β-sitosterol, Quassinoid; Ailantic acid; 1-carboxy-heneicosane pentadecanoate; hexyl pentaicosanote or pentyl pentaicosanoate. In an embodiment of the invention, the additional active ingredient is an antibacterial agent, a bactericide, an antiviral agent or a virucide. In an embodiment of the invention, the additional ingredient prevents a zoonotic disease. In some embodiments, the formulation further comprises one or more antiviral or virucidal ingredients. In some embodiments the additional antiviral or virucidal ingredient is administered separately. In some embodiments the additional antiviral or virucidal ingredient is administered to prevent viral infections such as lyssaviruses, including inter alia the rabies virus and Australian bat lyssavirus. In some embodiments, the additional antiviral or virucidal ingredient is administered to prevent viral infections and spreading of zoonotic viruses and other viruses. In some embodiments, the additional antiviral or virucidal ingredient is of natural herbal origin. In some embodiments, the additional antiviral or virucidal ingredient is from Datura.

In one embodiment, the present invention provides that the composition provides a synergistic combination of triptolide and/or its derivatives, salts, enantiomers, analogs or conjugates and gossypol and/or its derivatives, salts, enantiomers, analogs or conjugates.

In one embodiment, the synergistic effect relates to: birth control effect, contraception, sterility, infertility, or any combination thereof.

In one embodiment, the present invention provides that the composition is used as contraception and/or to sterilize and/or render infertile pests such as rodents. In one embodiment, the present invention provides that the composition is used as contraception and/or to sterilize and/or render infertile pet animals such as but not limited to dogs or cats. In one embodiment, the present invention provides that the composition is used as contraception and/or to sterilize and/or render infertile wild animals animals such as but not limited to kangaroos and coyotes.

In one embodiment, a composition as described herein inhibits mitochondrial lactate dehydrogenase (LDH)—C₄ and nuclear factor κB(NF-κB). In one embodiment, a composition as described herein reduces sperm production, function, quality or any combination thereof, in males. In one embodiment, a composition as described herein reduces fertility in females.

In one embodiment, a composition as described herein is used to inhibit aggressiveness in a subject in need thereof.

In one embodiment, a composition as described herein is used to enhance the quality of meat.

In one embodiment, a composition as described herein is used to inhibit inflammation and/or to relieve inflammatory symptoms in a patient in need thereof.

In one embodiment, suitable routes of administration of a composition as described herein, include oral, rectal, transmucosal, transnasal, intestinal or parenteral delivery, including intramuscular, subcutaneous and intramedullary injections as well as intrathecal, direct intraventricular, intravenous, intraperitoneal, intranasal, or intraocular injections.

In one embodiment, the composition is administered in a local rather than systemic manner, for example via injection of the preparation directly into a specific region of a patient's body.

Oral administration, in one embodiment, comprises a unit dosage form comprising tablets, capsules, lozenges, chewable tablets, suspensions, emulsions and the like. Such unit dosage forms comprise a safe and effective amount of the desired compound, or compounds, each of which is in one embodiment, from about 0.7 or 3.5 mg to about 280 mg/70 kg, or in another embodiment, about 0.5 or 10 mg to about 210 mg/70 kg. The pharmaceutically acceptable carriers suitable for the preparation of unit dosage forms for peroral administration are well-known in the art. In some embodiments, tablets typically comprise conventional pharmaceutically compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. In one embodiment, glidants such as silicon dioxide can be used to improve flow characteristics of the powder-mixture. In one embodiment, coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. In some embodiments, the selection of carrier components depends on secondary considerations like taste, cost, and shelf stability, which are not critical for the purposes of this invention, and can be readily made by a person skilled in the art.

In one embodiment, the oral dosage form comprises predefined release profile. In one embodiment, the oral dosage form of the present invention comprises an extended-release tablets, capsules, lozenges or chewable tablets. In one embodiment, the oral dosage form of the present invention comprises a slow-release tablets, capsules, lozenges or chewable tablets. In one embodiment, the oral dosage form of the present invention comprises an immediate release tablets, capsules, lozenges or chewable tablets. In one embodiment, the oral dosage form is formulated according to the desired release profile of the pharmaceutical active ingredient as known to one skilled in the art.

Peroral compositions, in some embodiments, comprise liquid solutions, emulsions, suspensions, and the like. In some embodiments, pharmaceutically acceptable carriers suitable for preparation of such compositions are well known in the art. In some embodiments, liquid oral compositions comprise from about 0.012% to about 0.933% of the desired compound or compounds, or in another embodiment, from about 0.033% to about 0.7%.

In some embodiments, compositions for use in the methods of this invention comprise solutions or emulsions, which in some embodiments are aqueous solutions or emulsions comprising a safe and effective amount of the compounds of the present invention and optionally, other compounds, intended for topical intranasal administration. In some embodiments, h compositions comprise from about 0.01% to about 10.0% w/v of a subject compound, more preferably from about 0.1% to about 2.0, which is used for systemic delivery of the compounds by the intranasal route.

In another embodiment, the pharmaceutical compositions are administered by intravenous, intra-arterial, or intramuscular injection of a liquid preparation. In some embodiments, liquid formulations include solutions suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intro-arterially, and are thus formulated in a form suitable for intro-arterial administration. In another embodiment, the pharmaceutical compositions are administered intramuscularly, and are thus formulated in a form suitable for intramuscular administration.

In another embodiment, the pharmaceutical compositions are administered topically to body surfaces, and are thus formulated in a form suitable for topical administration. Suitable topical formulations include gels, ointments, creams, lotions, drops and the like. For topical administration, the compounds of the present invention are combined with an additional appropriate therapeutic agent or agents, prepared and applied as solutions, suspensions, or emulsions in a physiologically acceptable diluent with or without a pharmaceutical carrier.

In one embodiment, pharmaceutical compositions of the present invention are manufactured by processes well known in the art, e.g., by means of conventional mixing, dissolving, granulating, dragee-making, levigating, emulsifying, encapsulating, entrapping or lyophilizing processes.

In one embodiment, pharmaceutical compositions for use in accordance with the present invention is formulated in conventional manner using one or more physiologically acceptable carriers comprising excipients and auxiliaries, which facilitate processing of the active ingredients into preparations which, can be used pharmaceutically. In one embodiment, formulation is dependent upon the route of administration chosen.

In one embodiment, injectables, of the invention are formulated in aqueous solutions. In one embodiment, injectables, of the invention are formulated in physiologically compatible buffers such as Hank's solution, Ringer's solution, or physiological salt buffer. In some embodiments, for transmucosal administration, penetrants appropriate to the barrier to be permeated are used in the formulation. Such penetrants are generally known in the art.

In one embodiment, the preparations described herein are formulated for parenteral administration, e.g., by bolus injection or continuous infusion. In some embodiments, formulations for injection are presented in unit dosage form, e.g., in ampoules or in multidose containers with optionally, an added preservative. In some embodiments, compositions are suspensions, solutions or emulsions in oily or aqueous vehicles, and contain formulatory agents such as suspending, stabilizing and/or dispersing agents.

The compositions also comprise, in some embodiments, preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfite and others; aromatic agents; viscosity adjustors, such as polymers, including cellulose and derivatives thereof; and polyvinyl alcohol and acid and bases to adjust the pH of these aqueous compositions as needed. The compositions also comprise, in some embodiments, local anesthetics or other actives. The compositions can be used as sprays, mists, drops, and the like.

In some embodiments, pharmaceutical compositions for parenteral administration include aqueous solutions of the active preparation in water-soluble form. Additionally, suspensions of the active ingredients, in some embodiments, are prepared as appropriate oily or water based injection suspensions. Suitable lipophilic solvents or vehicles include, in some embodiments, fatty oils such as sesame oil, or synthetic fatty acid esters such as ethyl oleate, triglycerides or liposomes. Aqueous injection suspensions contain, in some embodiments, substances, which increase the viscosity of the suspension, such as sodium carboxymethyl cellulose, sorbitol or dextran. In another embodiment, the is suspension also contain suitable stabilizers or agents which increase the solubility of the active ingredients to allow for the preparation of highly concentrated solutions.

In another embodiment, the active compound can be delivered, in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp, 317-327; see generally ibid).

In another embodiment, the pharmaceutical composition delivered in a controlled release system is formulated for intravenous infusion, implantable osmotic pump, transdermal patch, liposomes, or other modes of administration. In one embodiment, a pump is used (see Langer, supra; Sefton, CRC Crit. Ref. Biomed. Eng. 14:201 (1987); Buchwald et al., Surgery 88:50; (1980); Saudek et al., N. Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

In some embodiments, the active ingredient is in powder form for constitution with a suitable vehicle, e.g., sterile, pyrogen-free water-based solution, before use. Compositions are formulated, in some embodiments, for atomization and inhalation administration. In another embodiment, compositions are contained in a container with attached atomizing means.

In one embodiment, the preparation of the present invention is formulated in rectal compositions such as suppositories or retention enemas, using, e.g., conventional suppository bases such as cocoa butter or other glycerides.

In some embodiments, pharmaceutical compositions suitable for use in context of the present invention include compositions wherein the active ingredients are contained in an amount effective to achieve the intended, purpose. In some embodiments, a therapeutically effective amount means an amount of active ingredients effective to prevent, alleviate or ameliorate symptoms of disease or prolong the survival of the subject being treated.

In one embodiment, determination of a therapeutically effective amount is well within the capability of those skilled in the art.

The compositions also comprise preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; is antioxidants such as ascorbic acid, acetylcystine, sodium metabisulfote and others; aromatic agents; viscosity adjustors, such as polymers, including cellulose and derivatives thereof; and polyvinyl alcohol and acid and bases to adjust the pH of these aqueous compositions as needed. The compositions also comprise local anesthetics or other actives. The compositions can be used as sprays, mists, drops, and the like.

Some examples of substances which can serve as pharmaceutically-acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the Tween™ brand emulsifiers; wetting agents, such sodium lauryl sulfate; coloring agents; flavoring agents; tableting agents, stabilizers; antioxidants; preservatives; pyrogen-free water; isotonic saline; and phosphate buffer solutions. The choice of a pharmaceutically-acceptable carrier to be used in conjunction with the compound is basically determined by the way the compound is to be administered. If the subject compound is to be injected, in one embodiment, the pharmaceutically acceptable carrier is sterile, physiological saline, with a blood-compatible suspending agent, the pH of which has been adjusted to about 7.4.

In addition, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g. Tris-HCI, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, cellulose (e.g. Avicel™, RC-591), tragacanth and sodium alginate; typical wetting agents include lecithin and polyethylene oxide sorbitan (e.g. polysorbate 80). Typical preservatives include methyl paraben and sodium benzoate. In another embodiment, peroral liquid, compositions also contain one or more components such as sweeteners, flavoring agents and colorants disclosed above.

The compositions also include incorporation of the active material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

Also comprehended by the invention are particulate compositions coated with polymers e.g. poloxamers or poloxamines) and the compound coupled to antibodies directed against tissue-specific receptors, ligands or antigens or coupled to ligands of tissue-specific receptors.

In one embodiment, a composition as described herein is administered directly and/or locally into or to a reproductive organ. In one embodiment, a composition as described herein is contacted with a reproductive organ. In one embodiment, a reproductive organ comprises, testis, prostate gland, vas deferens, urethra, uterus, fallopian tubes, or ovary.

In one embodiment, provided herein a method for limiting birth rate comprising administering the composition described herein to a male, a female or both. In one embodiment, provided herein a method for controlling a population comprising administering the composition described herein to a male, a female or both. In one embodiment, provided herein a method for birth control, contraception, sterility, infertility or any combination thereof, comprising administering the composition described herein to a male, a female or both. In one embodiment, a male, a female or both is/are sexually matured. In one embodiment, the subject such as a male or a female is a human subject, an animal, a mammal, a bird, a reptile, or a pet.

In one embodiment, a method as described herein includes separate administration of: (a) triptolide and/or its derivatives, salts, enantiomers, analogs or conjugates; and (b) gossypol and/or its derivatives, salts, enantiomers, analogs or conjugates. In one embodiment, a method as described herein includes a kit comprising 2 separate compositions wherein the one composition comprises triptolide and/or its derivatives, salts, enantiomers, analogs or conjugates and the second composition comprises gossypol and/or its derivatives, salts, enantiomers, analogs or conjugates. In one embodiment, a method as described herein includes a kit comprising 2 separate compositions that are mixed prior to administration (10 hours to 15 seconds before administration). In one embodiment, a method as described herein includes a kit comprising 2 separate compositions wherein each composition is administered separately from the other composition.

The present invention relates to the surprising that both gossypol and/or gossypol acetic acid and triptolide inhibit Prostaglandin E₂ by inhibition of cytoplasmic phospholipase A₂ (cPLA₂) and Janus kinase (JA K)2 pathway, respectively. The combined inhibition of Prostaglandin E₂ results in the increases local blood flow and enhances vascular permeability in the reproductive organs, and in parallel, relieves inflammatory symptoms.

This discovery may improve the use of the formulation compared to the activity of each of the ingredients per se. The combination may provide more effective treatment in multiple ways and levels. Patients may benefit from the beneficiary effects of each component alone and from the additive and/or synergetic effects of the composition.

The patients may benefit from the cooperative effects triptolide and/or its derivatives, salts, enantiomers, analogs or conjugates and gossypol and/or its derivatives, salts, enantiomers, analogs or conjugates. In this case, the activity of the two components strongly dependents one on the other. For example, triptolide and/or its derivatives, salts, enantiomers, analogs or conjugates inhibit transcription factor nuclear factor κB(NF-κB) activation, resulting in poor sperm production. On the other hand, the sperm that is produced despite the activity of triptolide and/or its derivatives, salts, enantiomers, analogs or conjugates is subject to the activity of gossypol and/or its derivatives, salts, enantiomers, analogs or conjugates that inhibit lactate dehydrogenase (LDH). As mitochondrial LDH-C₄ presents only in the testes and sperm LDH-C₄ is essential for sperm function and the inhibition results in reduced sperm functions. This combined activity assures better efficacy.

In an embodiment of the invention, the inhibitor of LDH-C₄ is a condensed tannin. In an embodiment of the invention, the condensed tannin is gossypol and/or its derivatives, salts, enantiomers, analogs or conjugates. In an embodiment of the invention, the inhibitor of NF-κB is a diterpenoid epoxide. In an embodiment of the invention, the diterpenoid epoxide is triptolide and/or its derivatives, salts, enantiomers, analogs or conjugates. In some embodiments, triptolide and/or its derivatives, salts, enantiomers, analogs or conjugates and/or gossypol and/or its derivatives, salts, enantiomers, analogs or conjugates are extracted from a plant source, synthetic or semi-synthetic.

In some embodiments triptolide and/or its derivatives, salts, enantiomers, analogs or conjugates and/or gossypol and/or its derivatives, salts, enantiomers, analogs or conjugates are water soluble. In other embodiments they are in the form of an oil. In other embodiments they are in the form of oil-in-water. In other embodiments they are in the form of crystals.

In some embodiments triptolide and/or its derivatives, salts, enantiomers, analogs or conjugates and gossypol and/or its derivatives, salts, enantiomers, analogs or conjugates are administered separately. In some embodiments triptolide and/or its derivatives, salts, enantiomers, analogs or conjugates is administered first followed by gossypol and/or its derivatives, salts, enantiomers, analogs or conjugates administration. In some embodiments, gossypol and/or its derivatives, salts, enantiomers, analogs or conjugates is administered first followed by triptolide and/or its derivatives, salts, enantiomers, analogs or conjugates administration.

In some embodiments the formulation is administered orally for the purpose of population control at a daily triptolide and/or its derivatives, salts, enantiomers, analogs or conjugates dose is in the range of 20 μg/kg to 600 μg/kg. In some embodiments the formulation is administered orally for the purpose of population control at a daily triptolide and/or its derivatives, salts, enantiomers, analogs or conjugates dose is in the range of 50 μg/kg to 300 μg/kg. In some embodiments the formulation is administered orally for the purpose of population control at a daily triptolide and/or its derivatives, salts, enantiomers, analogs or conjugates dose is 60 to 150 μg/kg. In some embodiments the formulation is administered orally for the purpose of population control at a daily triptolide and/or its derivatives, salts, enantiomers, analogs or conjugates dose is 100 μg/kg. In some embodiments the formulation is administered orally for the purpose of population control at a daily gossypol and/or its derivatives, salts, enantiomers, analogs or conjugates dose is in the range of 10 μg/kg to 300 μg/kg. In some embodiments the formulation is administered orally for the purpose of population control at a daily gossypol and/or its derivatives, salts, enantiomers, analogs or conjugates dose is in the range of 20 μg/kg to 150 μg/kg. In some embodiments the formulation is administered orally for the purpose of population control at a daily gossypol and/or its derivatives, salts, enantiomers, analogs or conjugates dose is 20 to 50 μg/kg. In some embodiments the formulation is administered orally for the purpose of population control at a daily gossypol and/or its derivatives, salts, enantiomers, analogs or conjugates dose is 30 μg/kg. In some embodiments the concentration of triptolide and/or its derivatives, salts, enantiomers, analogs or conjugates is in a range of about 10% to 100% w/w in the oral formulation. In some embodiments the concentration of triptolide and/or its derivatives, salts, enantiomers, analogs or conjugates is in a range of about 0.0001% to 10% w/w in the oral formulation. In some embodiments the concentration of gossypol and/or its derivatives, salts, enantiomers, analogs or conjugates is in a range of about 10% to 100% w/w in the oral formulation. In some embodiments the concentration of gossypol and/or its derivatives, salts, enantiomers, analogs or conjugates is in a range of about 0.0001% to 10% w/w in the oral formulation. In some embodiments the ratio of triptolide and/or its derivatives, salts, enantiomers, analogs or conjugates and gossypol and/or its derivatives, salts, enantiomers, analogs or conjugates is 10:1 to 10:8. In some embodiments the ratio is 10:1 to 10:4. In some embodiments the ratio is 1:10. In some embodiments the ratio is 20:1 to 10:1. In some embodiments the ratio is 1:10 to 1:20.

In some embodiments the composition, or each ingredient, is administered by vapor. In some embodiments, the compositions, or each ingredient, may be added to food. In some embodiments the food is pet food. In some embodiments the food is edibles such as gummies. The composition, or each ingredient, may be packed in liposomes or emulsions of collagen, collagen peptides, nanospheres, micelles, polysaccharides or other components.

The absorption of the ingredients may be increased by combining the use of hostile biophysical environments with the use of penetrating agents, such as, but not limited to, oleoresin capsicum or its constituents or molecules containing heterocyclic rings to which hydrocarbon chains are attached.

The compositions of the present invention may include additional ingredients that are not physiologically active but serve to enhance the properties of the final composition. For example, the compositions of the present invention may include excipients such as lactose, dextrose, sucrose, sorbitol, mannitol, starch, gum acacia, calcium silicate, microcrystalline cellulose, polyvinylpyrrolidinone, cellulose, water, syrup, and methyl cellulose. The compositions of the present invention may include lubricating agents such as talc, magnesium stearate and mineral oil, wetting agents, emulsifying and suspending agents, preserving agents such as methyl- and propylhydroxybenzoates, sweetening agents or flavoring agents.

The compositions of the present invention may be formulated in any pharmaceutically acceptable topical vehicle that does not interact adversely with the active ingredients. Compositions of the present invention may be formulated in water or oil based topical vehicles. These compositions, in some embodiments, can include lanolin, aquaphor, methylcellulose and derivatives thereof, petroleum-based vehicles, Aloe vera and the like. In another embodiment, the compositions of the present invention are formulated in a topical, water-based vehicle containing Aloe vera and vitamin E.

The topical compositions of the present invention may include distilled water oil, stearic acid, an alcohol, an emulsifying wax, glycerin, palmitic acid, denatured alcohol, methyl salicylate, lecithin, sodium bicarbonate, ascorbyl palmitate, polysorbate, methylparaben, propylparaben, or any combination thereof. The topical compositions of the present invention may have a pH of between about 3 and about 8. According to embodiments topical composition of the present invention are in the form of an ointment, a cream, a lotion, an oil, a solution (in some embodiments an aqueous solution), an emulsion, a gel, a paste and a milk. In some embodiments the carrier is an aqueous-based carrier (such as a gel, oil-in water emulsion or oil-in water cream, aqueous solution, foam, lotion, spray).

As used herein, the singular forms “a”, “an”, and “the” include plural referents unless the context clearly dictates otherwise.

The terms “comprise,” “comprising,” “include,” “including,” “have,” and “having” are used in the inclusive, open sense, meaning that additional elements may be included.

The terms “or” or “and” as used herein should be understood to mean “and/or” unless the context clearly indicates otherwise.

As used herein, the terms “synergy” or “synergistic” or “synergetic effect” interchangeably refer to the combined effects of tat least wo active agents that are greater than their additive effects. Synergy can also be achieved by producing an efficacious effect with combined inefficacious doses of two active agents.

### EXAMPLES

### Example 1: Developmental and Reproduction Toxicity Study

Developmental and reproduction toxicity study, including pharmacokinetics, following repeatable oral administration of the test item (gavage) to Sprague Dawley Rats.

### Study Design

Rat age: 8 to 10 weeks at initiation
Total Number of Groups & Number of Animals per Group:
G1-Treatment. Vehicle Control: 5 males (M)+5 females (F)
G2-Treatment. 5M+5F (10 mg/kg bodyweight (bw)/day Triptolide+10 mg/kg bw/day Gossypol)
G3-Treatment. 5M+5F (10 mg/kg bw/day Triptolide)
G4-Treatment. 5M+5F (10 mg/kg bw/day Gossypol) G5-Treatment. 5M+5F (20 mg/kg bw/day Triptolide+30 mg/kg bw/day Gossypol)
G6-Treatment. 5M+5F (20 mg/kg bw/day Triptolide)
G7-Treatment. 5M+5F (30 mg/kg bw/day Gossypol)
G8-Pharmacokinetic group. 3M+3F (10 mg/kg bw/day Triptolide+10 mg/kg bw/day Gossypol)
Administration: Oral (Gavage)
Bleeding time points (G3): Sampling on day 1 & last day of sampling. 0.5, 2, 4, 8, 12 & 24 hours. 6 time points/day.
Sampling: Blood samples (200-400 μL/rat) are collected via orbital sinus in K2EDTA (20 μL of 200 mM/ml blood) coated tubes. Plasma separation using a refrigerated centrifuge and is stored at −70° C. before analysis.

Analysis: Plasma samples are processed and analyzed for triptolide and gossypol levels at each specified time-point using LC-MS/MS.

Endpoints: Fundamental PK parameters such as AUC(₀₋ₗₐₛₜ), AUCINF, T1/2, Vz, Tₘₐₓ and Cₘₐₓ are obtained from the non-compartmental analysis of the plasma/blood data using WinNonlin.

Dosing schedule [Treatment Period]: Males: At least for a period of 70 days (28 days during pre-mating+14 days during mating period+28 days post mating).

Females: At least for a period of 70 days and maximum 84 days including pre-mating, mating, gestation & lactation period (until lactation day 21)

Mating Procedure: 1:1 pairing within the same group until evidence of mating is confirmed or two weeks have elapsed.

### In-Life Observations: A. Systemic Toxicity End Points:

General clinical observations: daily.
Mortality and morbidity observations: twice daily
Body weight: weekly once during premating, mating and post-mating (if applicable).
Gestation body weight and feed consumption: gestation day 0, 7, 14 & 20.
Lactation body weight and feed consumption: lactation day 1, 4, 7, 13 & 14 (at termination).
Feed consumption: weekly once coinciding with body weight, except during mating.
Gestation feed consumption: gestation day 0 to 7, 7 to 14 & 14 to 20.
Lactation feed consumption: lactation day 1 to 4, 4 to 7, 7 to 13.
   b. Reproduction Toxicity End Points:
Oestrus cycle evaluations:
All females during pre-mating, cohabitation period until confirmation of mating.
Reproductive performance:
Mating index, fertility index, pre-coital interval, gestation length, gestation index, pregnancy/parturition index, post-implantation loss and post-natal loss.
Live birth index, pup survival index, sex ratio per litter.
   c. Developmental Toxicity End Points:
Daily pup observations until termination during postnatal period.
Individual pup weight on postnatal days (PND) 1, 4, 7 and 13.
Post-mortem observations-Termination day:
Males: after at least 70 days of treatment;
Females: on lactation day 21 (littered dams)
Pups: on PND 21 (all surviving pups).

### Observations:

a. Gross pathological examination [adults & pups]: detailed gross necropsy, with careful examination of external surface of the body, all orifices, the cranial, thoracic and abdominal cavities and their contents as well as organs and tissues of each animal with “special emphasis on reproductive organs”.
b. Organ Weights: epididymites, ovaries, prostate, seminal vesicles with coagulation glands, testes, uterus with cervix.
c. Gross lesions (if any): gross lesions if any found during the conduct of necropsy will be preserved and evaluated histopathologically if required.
d. Histopathology: initially for all reproductive organs/tissues collected from control & high dose group and later will be extended to lower dose group animals in case of treatment related effects noted in high dose level.
Results: in G-5 no pregnancy is observed, while in G-1 90% of females got pregnant.

### Example 2: Triptolide/Gossypol Treatment of GT1-1 and GC-1Spg Cell Lines

Aim: To test Triptolide/Gossypol effect on expressions/regulations of cPLA2, Prostaglandin E2, JAK2, NF-κB pathway and LDHC4.

Experimental design: GT1-1 and GC-1spg cell lines are cultured separately in 6-well plate to about 80% confluence, and add triptolide and gossypol according to a dosage regimen matrix. After 24 hrs of further culture, cells are collected for following assays.

Assay: 1) cPLA₂ enzymatic activity assay in GT1-1 cell lysate will be measured with EnzChek™ Phospholipase A₂ Assay Kit according to kit manual; 2) Prostaglandin E₂ amount in GT1-1 cell lysate will be measured with commercial ELISA kit according to kit manual; 3) As for JAK2 pathway, both total and phosphorylated JAK2 and STAT3 proteins in GT1-1 cell lysate will be detected by western blot assay; 4) As for NF-κB pathway, both total and phosphorylated NF-κB p65 proteins in GT1-1 cell lysate will be detected by western blot assay; 5) LDHC4 protein in GC-1spg cell lysate will be detected by western blot assay.

Results: The combined effect of triptolide and gossypol treatment result in significant less activity of the cells.

### Example 3: Reducing Fertility in Cats

The study includes 5 groups: three groups with 3 different concentrations of the active substances, a group with a reduced administration frequency and a placebo group.

Each group (including the in vivo group) will include 36 individuals, plus 4 individuals (about 10%) for unexpected behavior, a total of 200 animals.

During the experiment, animals stay in the pen for 3 months. The monitoring of the animals continues for 24 months. Only mature and healthy non-sterilized/neutered cats/cats participate in the research on caged animals. All the cats are tagged with an electronic chip.

Behavioral tests: Reproductive behavior (libido, estrus, mating, pregnancy, calving), sexual behavior, clinical tests, including ultrasound and gynoscopy, sperm test (are taken using an artificial vagina), prostate, testicles, ovaries, uterus, udders.

Laboratory tests: testosterone, LH (Luteinizing hormone), FSH (follicle-stimulating hormone), AMH (Anti-Müllerian hormone), progesterone, estrogen, ejaculation quality (volume, concentration of sperm cells, % of sperm cells with motility and with progressive motility, morphology, differential count of the defects in the sperm and their essence, cytological examination to find abnormal cells (blood, various white blood cells, bacteria, etc.)), Histopathology and vaginal surface.

Blood samples are taken and the serum is separated and kept in a deep freeze −80° C. until sex hormones and other measurable factors are tested.

At the end of the study, each animal that is spayed/neutered and only its gonads undergo a histopathological examination.

Results: In cats treated with the combination of triptolide and gossypol, at all concentrations, fertility is reduced by 78%.

1. A composition comprising: (a) triptolide, a triptolide prodrug, a pharmaceutically acceptable salt of triptolide, a pharmaceutically acceptable salt of a triptolide prodrug, or any combination thereof; and (b) gossypol, a pharmaceutically acceptable salt of gossypol, or a combination thereof.

2. The composition of claim 1, wherein said triptolide, said triptolide prodrug, said pharmaceutically acceptable salt of triptolide, said pharmaceutically acceptable salt of said triptolide prodrug, said gossypol, said pharmaceutically acceptable salt of gossypol, or any combination thereof is extracted from a plant.

3. The composition of claim 1, wherein said triptolide, said triptolide prodrug, said pharmaceutically acceptable salt of triptolide, said pharmaceutically acceptable salt of said triptolide prodrug, said gossypol, said pharmaceutically acceptable salt of gossypol, or any combination thereof is synthetic or semi-synthetic.

4. The composition according to claim 1, further comprising at least one additional active ingredient.

5. The composition of claim 4, wherein said at least one additional active ingredient comprises an antibacterial agent, or an antiviral agent.

6. The composition according to claim 1, wherein said composition is in an oral dosage form or an injectable dosage form.

7. A kit comprising: a first composition comprising: triptolide, a triptolide prodrug, a pharmaceutically acceptable salt of triptolide, a pharmaceutically acceptable salt of a triptolide prodrug or any combination thereof; and a second composition comprising gossypol, a pharmaceutically acceptable salt of gossypol, or a combination thereof.

8. A method for any one of limiting birth, inducing contraception, inducing abortion, and inducing sexual sterility or infertility in a subject in need thereof, the method comprising administering to said subject, a therapeutically effective amount of the composition of claim 1, thereby limiting birth, inducing contraception, inducing abortion, or inducing sexual sterility or infertility, in the subject.

9. The method of claim 8, wherein said subject is a mammal, a reptile, or a bird.

10. The method of claim 8, wherein said administering comprises contacting a reproductive organ of said subject with said composition.

11. The method of claim 8, wherein said first composition and said second composition are administered separately or sequentially.

12. The method of claim 8, wherein said first composition and said second composition are administered concomitantly.

13. The method of claim 8, wherein said administering comprises orally administering.

14. The method of claim 8, wherein said administering comprises administering to a reproductive organ of said subject.

15. The method of claim 8, wherein said subject is a male.

16. The method of claim 8, wherein said subject is a female.

17. The method of claim 8, wherein said subject is a mammal or a bird.
